(19) United States
(12) Patent Application Publication
(10) Pub. No.: US 2010/0236707 A1
(43) Pub. Date: Sep. 23, 2010

(54) METHOD OF BONDING

(76) Inventors: Katia Studer, Rixheim (FR); Tunja Jung, Rheinfelden-Herten (DE); Kurt Dietliker, Allschwil (CH)

Correspondence Address: BASF Corporation;Patent Department, 500 White Plains Road, P.O. Box 2005, Tarrytown, NY, 10591, US

(21) Appl. No.: 12/399,146

(22) PCT Filed: Jul. 9, 2007
(86) PCT No.: PCT/EP2007/056917

§ 371 Date: Jan. 8, 2009

(30) Foreign Application Priority Data
Jul. 17, 2006 (EP) 06117329.0

(51) Int. Cl. B29C 65/14; C07C 221/00

(52) U.S. Cl. 156/275.5; 564/344

(57) ABSTRACT

The invention relates to a first method of bonding a first substrate to a second substrate, comprising the steps of a) applying an UV-curable adhesive resin composition comprising a photolatent base to at least one transparent surface of at least one of said first and second substrates, b) bringing said first and second substrates together with said adhesive composition there between, c) exposing said adhesive composition to actinic radiation to effect curing or alternatively to a second method of bonding a first substrate to a second substrate, comprising the steps of a) applying a UV-curable adhesive resin composition comprising a photolatent base to one surface, b) exposing said adhesive composition to actinic radiation to effect curing, c) bringing said first and second substrates together with said adhesive composition there between.

The invention relates to a method of bonding a first substrate to a second substrate by means of a radiation-curable adhesive composition comprising a photolatent base.

Finding the right balance between formulation stability and reactivity at low temperature under ambient atmosphere is a major concern for most of the adhesive systems.

2K adhesives generally based on NCO/OH, NCO/SH and epoxy/amines require the addition of a catalyst to cure in a short period of time at low temperature, which shortens the pot life of the formulation.

1K adhesives, generally based on epoxies or moisture-cured isocyanates also require the presence of a catalyst in order to speed up the curing at low temperature which here again shows the major drawback of negatively affecting the formulation stability. Other 1K adhesive formulations such as cyanoacrylates or silane-modified polymer also react with ambient humidity at room temperature, giving formulations of poor stability.

Anaerobic adhesives, usually containing small amounts of peroxide and accelerators, remain liquid as long as they are in contact with atmospheric oxygen, but cure generally within a few hours once placed in an inert atmosphere or in contact with a metallic surface. For storage, it is necessary to fill half of the flask with air to avoid any premature gelling.

UV-curable adhesive systems react at room temperature after light activation and remain stable in the dark. Cure is however either partly inhibited by dissolved and diffusing oxygen in the case of acrylates or sensitive to moisture in the case of epoxies. The choice of UV-curable groups is also limited to epoxies or double bonds so far.

Hotmelts or plastisol have to be processed at high temperature (between 120° C. and 240° C.) prior to use, which limits the application range to substrates which are resistant to heat.

The European Patent EP898202B1 (Ciba) describes base catalyzed curable compositions comprising alpha-aminoketone compounds as latent base and its use in adhesives based on epoxide resins (see paragraph 0002). A suitable resin is for example a polyacrylate with 3-5% carboxylic function and an epoxy phenol novolac (see Ex. 1).

The International application WO01/92362 (AKZO) relates to a photoactivatable coating composition comprising at least one polyisocyanate and at least one compound comprising isocyanate reactive groups. The isocyanate reactive groups comprise at least one thiol group and the photoinitiator is a photolatent base. The coating compositions show particular utility as clear coats, base coats, pigmented top coats, primers, and fillers. WO01/92362 appears to provide no suggestion for use of any such copolymer as or in an adhesive.

The International application WO06008251 (Ciba) describes a process for the application of a photolatent base wherein an adhesive comprising said catalyst is subjected to irradiation before being further processed. However, the adhesive properties are not sufficient.

It has now been found that exposure after the lamination results in faster curing. Furthermore, the diamine catalyst usually present in OH/NCO or SH/NCO systems can be replaced by the latent base.

Thus, the present invention relates to a method of bonding a first substrate to a second substrate, comprising the steps of

a) applying an UV-curable adhesive resin composition comprising a photolatent base to at least one transparent surface of at least one of said first and second substrates,
b) bringing said first and second substrates together with said adhesive composition there between,
c) exposing said adhesive composition to actinic radiation to effect curing.

A further subject of the invention is a method of bonding a first substrate to a second substrate, comprising the steps of

a) applying an UV-curable adhesive resin composition comprising a photolatent base to one surface,
b) exposing said adhesive composition to actinic radiation to effect curing
c) bringing said first and second substrates together with said adhesive composition there between.

### DEFINITIONS

### Substrates

In one embodiment of the invention at least one substrate has to be transparent and is preferably selected from glass, fiberglass, ceramic material, paper and plastics such as polyester, polyethylene, polycarbonate, polyethylene, polypropylene, polystyrene, polyvinylchloride, rubbers and the like.

The other substrate (or both substrates in another embodiment of the invention) is for example non-transparent and may be in addition metal, ceramic, wood, rubber, non-transparent plastic, e.g. colored plastic, as described above, and the like.

### UV-Curable Adhesive

UV-curable adhesives are preferably OH/NCO or SH/NCO systems. These adhesives are produced by the condensation reaction of an organic polyisocyanate with an active hydrogen-containing compound.

The isocyanate compound may be any aromatic, aliphatic, cycloaliphatic, acryl aliphatic, or heterocyclic isocyanate or polyisocyanate, and the prepolymers or mixtures thereof. The term “polyisocyanates” includes diisocyanates, triisocyanates, tetraisocyanates, etc., and mixtures thereof. Suitable isocyanate compounds are for example commercially available from Bayer under the name Desmodur® or from Rhodia under the trade name Tolonate®.

The active hydrogen containing compound has functional groups which are for example selected from the group consisting of —COOH, —OH, —NH₂, —NH—, —CONH₂, —SH, and —CONH—. Preferably the active hydrogen containing compound is OH or SH resulting in OH/NCO and SH/NCO resins.

For example the active hydrogen and/or isocyanate component can be blocked to increase the formulation shelf life. The blocking agent is released under the action of heat and/or of the active catalyst. Examples of suitable blocked components are known to the person skilled in the art.

OH/NCO systems are known as polyurethane adhesives. Polyurethane adhesives are for example one-component polyurethane adhesives (1K PU adhesives) or two-component polyurethane adhesives (1K PU adhesives).

Polyester polyols and polyether polyols preferably used as active hydrogen containing compound in OH/NCO resins are for example commercially available materials.

Suitable polyesterpolyols are commercially available, for example under the trade name Desmophen® and Baycoll®.

Optionally a multifunctional aliphatic amine chain extender is present in the adhesive composition. Examples of additional chain extenders in the binder are given in “Formulierung von Kleb- and Dichtstoffen, B. Müller, W. Rath, Vincentz Network, Hannover, 2004, p. 121”, e.g. diols or triols of relatively low molecular weight such as 1,2-ethandiol, 1,4-butandiol, 1,6-hexandiol, 2-ethyl-1,3-hexandiol and 1,4-cyclohexandimethanol. Such compounds further include ethylene diamine, 1,4-butanediamine, isophorene diamine, triethylenetetraamine, and triethylene oxide diamine.

Furthermore desiccants may be present such as for example Baylith L.

Suitable thiol group containing compounds are those as described in WO01/92362. As disclosed therein the most preferred thiol-functional compounds are pentaerythritol tetrakis(3-mercaptopropionate) and 3-mercaptopropionate.

### The Photolatent Base

Any photolatent bases possessing suitable basicity are applicable in the context of the present invention.

Thus, the photolatent base is for example a compound of the formula (I) as disclosed in EP970085 (Ciba) or WO03/033500 (Ciba) (the disclosure of these documents hereby is incorporated by reference)

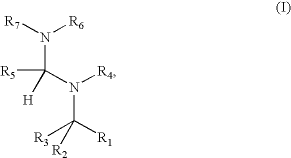

in which
R₁ is phenyl, biphenylyl, naphthyl, anthryl or anthraquinonyl all of which are unsubstituted or substituted by one or more of the substituents C₁-C₄-alkyl, C₂-C₄-alkenyl, CN, OR₁₀, SR₁₀, COOR₁₂, halogen or a substituent of structure (II)

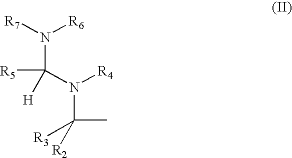

or
R₁ is a substituent of formula (IIIa) or (IIIb)

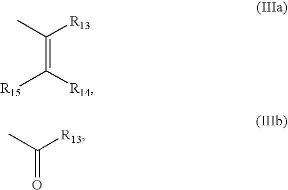

in which
R₁₃ is phenyl, biphenylyl, naphthyl, anthryl or anthraquinonyl all of which are unsubstituted or substituted by one or more of the substituents C₁-C₄-alkyl, C₂-C₄-alkenyl, CN, OR₁₀, SR₁₀, COR₁₁, COOR₁₂, or halogen;
R₁₄ is hydrogen
R₁₅. is hydrogen or C₁-C₄-alkyl;
R₂ and R₃ independently of each other are hydrogen or C₁-C₆-alkyl;
R₄ and R₆ together form a C₂-C₆-alkylene bridge that is unsubstituted or substituted by one or more C₁-C₄-alkyl; or
R₅ and R₇, together form a C₂-C₆-alkylene bridge that is unsubstituted or substituted by one or more C₁-C₄-alkyl;
R₁₀, R₁₁ and R₁₂ independently of each other are hydrogen or C₁-C₆-alkyl.

Especially preference is given to a compound of the formula (I)

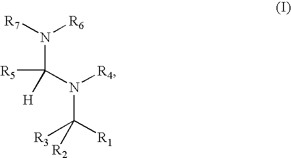

in which
R₁ is phenyl, biphenylyl or naphthyl all of which are unsubstituted or substituted by one or more of the substituents C₁-C₄-alkyl, CN, OR₁₀, SR₁₀, COOR₁₂, or a substituent of structure (II)

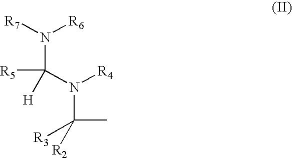

or
R₁ is a substituent of formula (III)

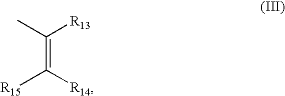

in which
R₁₃ is phenyl, biphenylyl or naphthyl all of which are unsubstituted or substituted by one or more of the substituents C₁-C₄-alkyl, CN, OR₁₀, SR₁₀, or COOR₁₂;
R₁₄ and R₁₅ are hydrogen;
R₂ and R₃ independently of each other are hydrogen or C₁-C₆-alkyl;
R₄ and R₆ together form a C₃-alkylene bridge that is unsubstituted or substituted by one or more C₁-C₄-alkyl; or
R₅ and R₇, together form a C₃-C₅-alkylene bridge that is unsubstituted or substituted by one or more C₁-C₄-alkyl;
R₁₀ and R₁₂ independently of each other are hydrogen or C₁-C₆-alkyl.

Preferred are compounds like

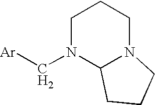

wherein Ar phenyl, biphenylyl or naphthyl all of which are unsubstituted or substituted by one or more of the substituents C₁-C₄-alkyl, CN, OH, O—C₁-C₆alkyl, SH, S—C₁-C₆alkyl, COOH, COO—C₁-C₆alkyl.

An especially preferred example is 5-benzyl-1,5-diazabicyclo[4.3.0]nonane

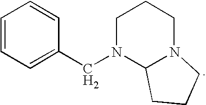

Concerning the compounds of formula I wherein R₁ is a substituent of formula III, examples are

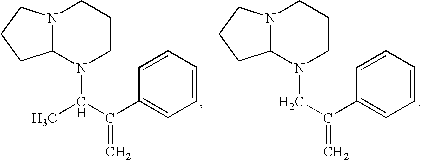

Furthermore the photolatent base to be used in the present method is a compound of the formula IV as disclosed in EP898202 (Ciba), (the disclosure of said document hereby is incorporated by reference).

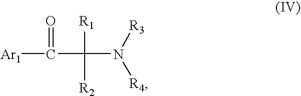

in which
Ar₁ is an aromatic radical of formula V or VIII

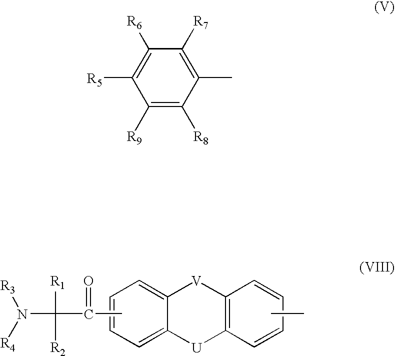

### U is —N(R₁₇)—;

V has the meaning of U or is a direct bond;
R₁ and R₂ are each independently of each other
a) C₁-C₁₂-alkyl, which is unsubstituted or substituted by OH, C₁-C₄-alkoxy, or SH,
b) a radical of formula

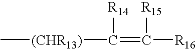

or
c) a radical of formula

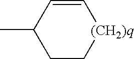

in which q is 0, or 1, or
d) a radical of formula

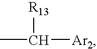

e) phenyl which is unsubstituted or substituted C₁-C₄-alkyl, or R₁ and R₂ together are unbranched or branched C₄-C₆-alkylene or C₃-C₅-oxaalkylene;
   Ar₂ is phenyl which is unsubstituted or substituted by halogen, OH, C₁-C₁₂-alkyl, or is substituted by C₁-C₄-alkyl, which is substituted by OH, halogen, C₁-C₁₂-alkoxy, —COO(C₁-C₄-alkyl), —CO(OCH₂CH₂)ₙOCH₃ or —OCO(C₁-C₄-alkyl), or the radical phenyl, is substituted by C₁-C₄-alkoxy, —(OCH₂CH₂)ₙOH, or —(OCH₂CH₂)ₙOCH₃;
   n is 1-5;
   R₃ is C₁-C₄-alkyl, C₂-C₄-alkyl which is substituted by —OH, —C₁-C₄-alkoxy, —CN, or —COO(C₁-C₄-alkyl), or R₃ is C₃-C₅-alkenyl, or phenyl-C₁-C₃-alkyl-;
   R₄ is C₁-C₄-alkyl, C₂-C₄-alkyl which is substituted by —OH, —C₁-C₄-alkoxy, —CN, or —COO(C₁-C₄-alkyl), or R₃ is C₃-C₅-alkenyl, or phenyl-C₁-C₃-alkyl-, or R₃ and R₄ together are C₃-C₇-alkylene which can be interrupted by —O—, or —S—;
   R₅, R₆, R₇, R₈ and R₉ are each independently of one another hydrogen, halogen, C₁-C₁₂-alkyl, phenyl, benzyl, benzoyl, or a group —OR₁₇, —SR₁₈, —N(R₁₉)(R₂₀), or

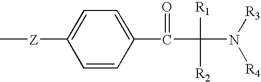

### Z is —O—, —S—, —N(R₁₁)—, —N(R₁₁)—R₁₂—N(R₁₁)— or

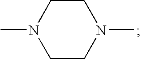

R₁₁ is C₁-C₄-alkyl;
R₁₂ is unbranched or branched C₂-C₁₆-alkylene which can be interrupted by one or more —O— or —S—;
R₁₃ is hydrogen or C₁-C₄-alkyl;
R₁₄, R₁₅ and R₁₆ are each independently of one another hydrogen or C₁-C₄-alkyl, or
R₁₄ and R₁₅ together are C₃-C₄-alkylene;
R₁₇ is hydrogen, C₁-C₁₂-alkyl, C₃-C₆-alkenyl, C₂-C₆-alkyl which is substituted by —CN, —OH or —COO(C₁-C₄-alkyl);
R₁₈ is hydrogen, C₁-C₁₂-alkyl, C₃-C₆-alkenyl, C₂-C₁₂-alkyl which is substituted by —OH, —CN, —COO(C₁-C₄-alkyl);
R₁₉ and R₂₀ are each independently of the other C₁-C₆-alkyl, C₂-C₄-hydroxyalkyl, C₂-C₁₀-alkoxyalkyl, C₃-C₅-alkenyl, phenyl-C₁-C₃-alkyl, phenyl which is unsubstituted or substituted by C₁-C₄-alkyl or C₁-C₄-alkoxy, or R₁₉ and R₂₀ are C₂-C₃-alkanoyl or benzoyl, or R₁₉ and R₂₀ are —O(CO—C₁-C₈)ₒ—OH;
o is 1-15;
or R¹⁹ and R₂₀ together are C₄-C₆-alkylene which can be interrupted by —O—, —N(R₂₂)— or —S—, or R₁₉ and R₂₀ together are C₄-C₆-alkylene which can be substituted by hydroxyl, C₁-C₄-alkoxy or —COO(C₁-C₄-alkyl);
R₂₂ is C₁-C₄-alkyl, phenyl-C₁-C₃-alkyl, —CH₂CH₂—COO(C₁-C₄-alkyl), —CH₂CH₂CN, —CH₂CH₂—COO(CH₂CH₂O)_{q}—H or

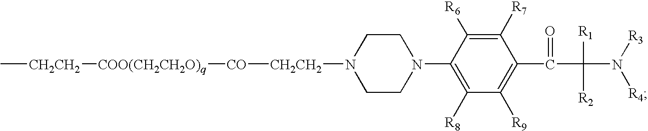

and
q is 1-8.

Preferred are compounds of the formula (IV)

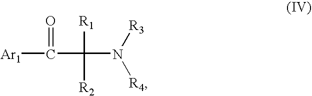

in which
Ar₁ is an aromatic radical of formula V or VIII

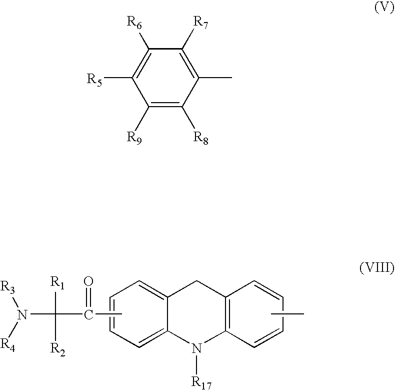

R₁ and R₂ are each independently of each other
a) C₁-C₆-alkyl, which is unsubstituted or substituted by OH, C₁-C₄-alkoxy, or SH,
b) a radical of formula

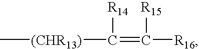

c) a radical of formula

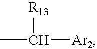

or R₁ and R₂ together are unbranched or branched C₄-C₆-alkylene;
Ar₂ is phenyl which is unsubstituted or substituted by OH, C₁-C₆-alkyl, or is substituted by C₁-C₄-alkyl, which is substituted by OH, C₁-C₄-alkoxy, —COO(C₁-C₄-alkyl), or the radical phenyl is substituted by C₁-C₄-alkoxy, —(OCH₂CH₂)ₙOH, or —(OCH₂CH₂)ₙOCH₃;
n is 1-3;
R₃ is C₁-C₄-alkyl, C₂-C₄-alkyl which is substituted by —OH, —C₁-C₄-alkoxy, —CN, or —COO(C₁-C₄-alkyl), or R₃ is C₃-alkenyl, or phenyl-C₁-alkyl-;
R₄ is C₁-C₄-alkyl, C₂-C₄-alkyl which is substituted by —OH, —C₁-C₄-alkoxy, —CN, or —COO(C₁-C₄-alkyl), or R₃ is C₃-alkenyl, or phenyl-C₁-alkyl-, or R₃ and R₄ together are C₄-C₅-alkylene which can be interrupted by —O—;
R₇ and R₈ are hydrogen;
R₅, R₆, and R₉ are each independently of one another are hydrogen, halogen, C₁-C₄-alkyl, phenyl, benzyl, or a group —OR₁₇, —SR₁₈, —N(R₁₉)(R₂₀);
R₁₃ is hydrogen or methyl;
R₁₄, R₁₅ and R₁₆ are each independently of one another hydrogen or methyl;
R₁₇ is hydrogen, C₁-C₆-alkyl, C₃-C₆-alkenyl;
R₁₈ is hydrogen, C₁-C₄-alkyl, C₃-C₆-alkenyl;
R₁₉ and R₂₀ are each independently of the other C₁-C₆-alkyl, C₂-C₄-hydroxyalkyl, C₂-C₁₀-alkoxyalkyl, C₃-C₅-alkenyl, phenyl-C₁-C₃-alkyl, or R₁₉ and R₂₀ are —O(CO—C₁-C₈)ₒ—OH or R₁₉ and R₂₀ together are C₄-C₅-alkylene which can be interrupted by —O—, —N(R₂₂)— or —S—;
o is 1-10;
R₂₂ is C₁-C₄-alkyl, phenyl-C₁-C₃-alkyl, —CH₂CH₂—COO(C₁-C₄-alkyl), —CH₂CH₂—COO(CH₂CH₂O)_{q}—H or

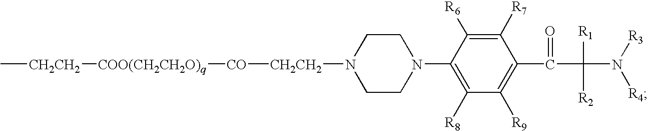

and
q is 1-6.

In the context of the present invention all defined alkyl, alkylene and oxaalkylene, uninterrupted or interrupted, are meant to be linear (unbranched) or branched, even if not expressly stated in the definition as such.

Examples of specific compounds are:

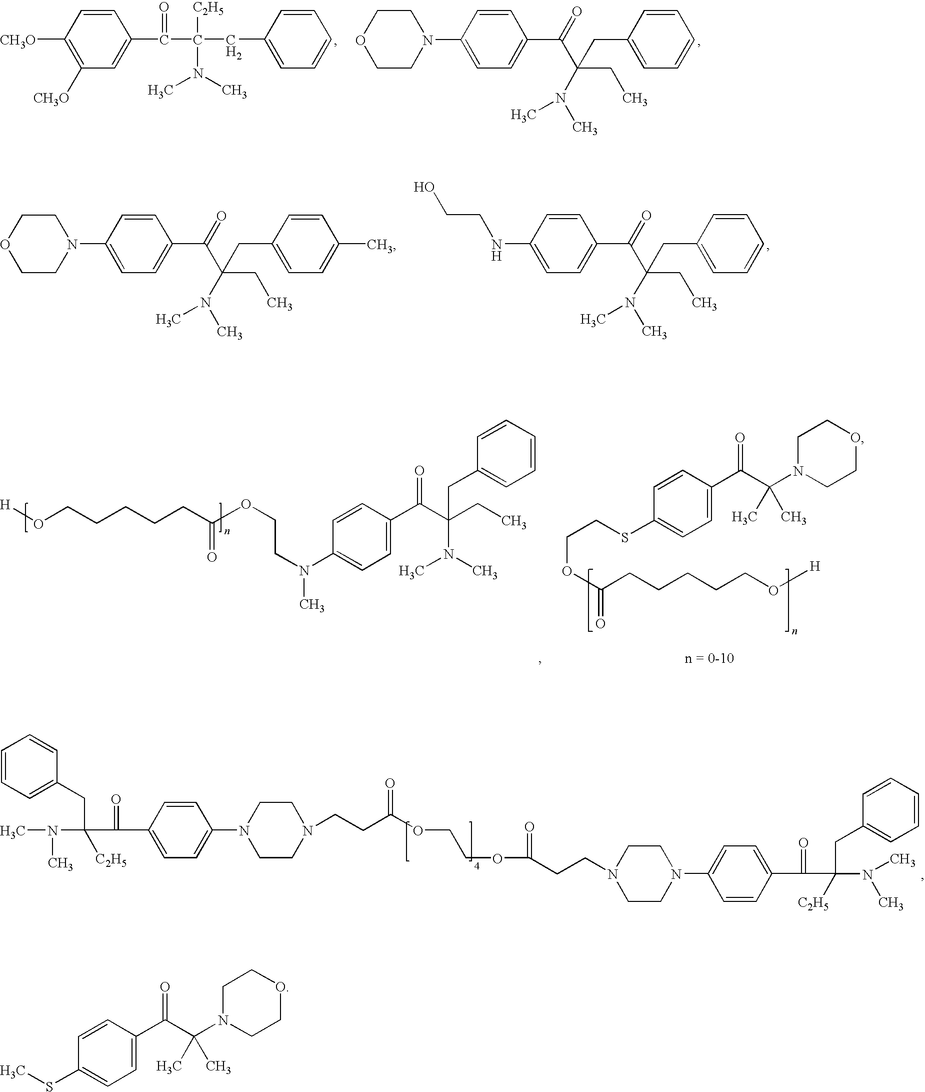

### Optional Ingredients

The adhesive composition of the invention optionally also contains other compounds such as antioxidants (especially Hals-compounds), filler resins, thickeners, fluidity adjusting agents, plasticizers, defoaming agents and the like, known in the art for such compositions.

### Amounts

The photolatent base is for example used in an amount between 0.01 to 10 wt. % on solid curable material, preferably 0.05 to 5 wt. %, more preferably 0.05 to 3 wt. %.

The polyisocyanate may be mixed with the compound having a OH/SH function by any suitable technique known in the art.

The pre-polymerized adhesives containing isocyanate and the reactive groups (protected or not) are for example processed at high temperature and coated onto the substrate following the hotmelt process, afterwards full cure is achieved by an additional curing step involving the reactive groups, which is realized by photoactivation of the photolatent catalyst (i.e. the photolatent base compound).

Hotmelt adhesives are interesting as pressure sensitive adhesives (PSA) and suitable to replace the use of solvent based compositions, which from an environmental point of view are disadvantageous. The hotmelt extrusion process necessitates high application temperatures in order to achieve the high flow viscosity. The compositions of the present invention comprising reactive groups are suitable as crosslinkers in the preparation of a hotmelt coating, where the crosslinkers enter into a chemical reaction with the functional comonomers of the (meth)acrylate PSA. After the coating operation, the PSAs are first crosslinked thermally, or, implementing the dual crosslinking mechanism, the PSA is subsequently crosslinked with UV light. UV crosslinking irradiation for example is effected by means of shortwave ultraviolet radiction in a wavelength range from 200 to 400 nm, depending on the UV photoinitiator. Such systems and processes are for example described in US 2006/0052472.

### Radiation

Suitable radiation is present, for example, in sunlight or light from artificial light sources. Consequently, a large number of very different types of light source are employed. Both point sources and arrays (“lamp carpets”) are suitable. Examples are carbon arc lamps, xenon arc lamps, medium-, high- and low-pressure mercury lamps, possibly doped with metal halide (metal-halogen lamps), microwave-excited metal vapour lamps, excimer lamps, superactinic fluorescent tubes, fluorescent lamps, argon incandescent lamps, electronic flashlamps, photographic floodlamps, light emitting diodes (LED, OLED), electron beams and X-rays, produced by means of synchrotrons or laser plasma. Fluorescent lamps are preferred which produce UV A light. A suitable lamp is, for example the actinic blue lamp Philips TL20W/05 which emits light between 300 nm and 400 nm.

### Thickness

The thickness of the formed adhesive film is preferably from 5 to 200 μm.

### Advantage

Employing photolatent bases to trigger the cure of adhesives using light would allow a fast cure at low temperature while keeping the adhesive formulation stable in the dark. Cure is not inhibited by oxygen or moisture.

The examples which follow illustrate the invention in more detail, without restriciting the scope said examples only. Parts and percentages are, as in the remainder of the description and in the claims, by weight, unless stated otherwise. Where alkyl radicals having more than three carbon atoms are referred to in the examples without any mention of specific isomers, the n-isomers are meant in each case.

The following photolatent base compounds are used in the examples:

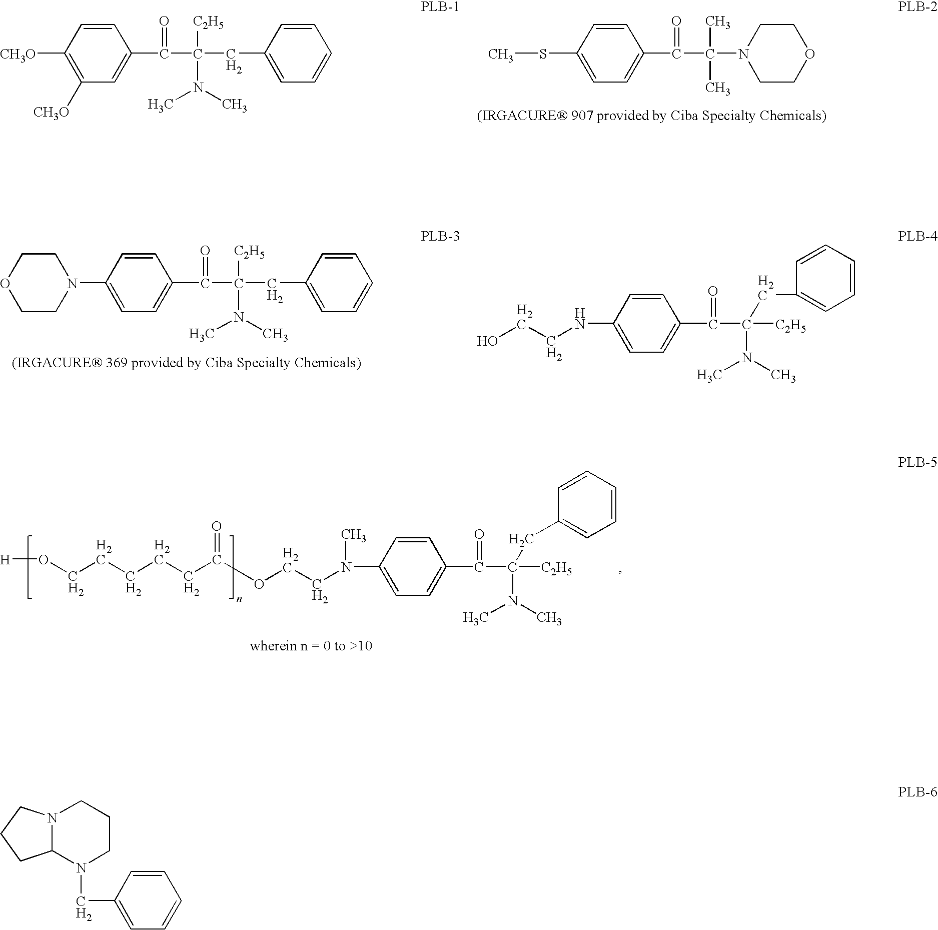

### EXAMPLE 1

### SH/NCO-System

| % weight | Product | Description |
|---|---|---|
| 0.2 g | PLB-1 | Photolatent base |
| 13.3 g | Trimethylolpropane tris(3 mercapto- | Thiol component |
| | propionate) (Aldrich) | |
| 25 g | Tolonate HDT LV (Rhodia) | Isocyanate component |

PLB-1 is dissolved in the thiol component and isocyanate is added shortly before application. A 120 μm thick film is applied onto a glass plate (plate A). A second glass plate (plate B), not coated with the adhesive, is pressed on plate A. After laminating plate A and plate B, the system is irradiated for 5 minutes under a fluorescent lamp (Philips TL20W/05). After irradiation, it is no more possible to separate both glass plates. As a comparison, the same experiment is repeated with a system stored for 5 minutes in the dark instead of being irradiated. Both glass plates can be easily detached, the formulation being still liquid.

### EXAMPLE 2

### OH/NCO System

Component A (OH component)

| Weight (g) | Product | Description |
|---|---|---|
| 104 | Desmophen 5034 BT | Trifunctional polypropylene |
| | (Bayer AG) | ether polyol |
| 181 | Baycoll VP KA 8576 | Polyester-polyol |
| | (Bayer AG) | |
| 5 | Baylith L (Bayer AG) | Desiccant |
| 1 | Isophorene diamines (Bayer | Chain extender |
| | AG) | |

A formulation having the following composition is prepared:

| Weight (g) | Product | Description |
|---|---|---|
| 0.3 | PLB-1 | Photolatent base |
| 2 | Butyl Acetate (Aldrich) | Solvent |
| 5 | Component A | OH component |
| 5 | Desmodur E 23 (Bayer AG) | Aromatic polyisocyanate pre- |
| | | polymer |

A 10 μm thick film of the above mentioned formulation is laminated between two BaF₂ crystals and further exposed to UV light (medium pressure mercury lamp AETEK International, one pass at a belt speed of 5 m/min with 2 lamps at 80 W/cm). The reaction is monitored by IR spectroscopy by following the decrease of the isocyanate peak at 2271 cm⁻¹ at room temperature after UV-exposure. The lower the NCO content, the better are the adhesive properties of the film.

As a comparison, the same experiment is performed for a non-laminated film and for a non-exposed film. Results are given in the table below, and clearly show that the curing of the adhesive is faster for the exposed and laminated film.

| | NCO content (%) | | |
|---|---|---|---|
| Time at RT | UV-exposed | | UV-exposed |
| (min) | Laminated | Non-irradiated | Non-laminated |
| 0 | 66 | 100 | 84 |
| 5 | 49 | 85 | 66 |
| 9 | 40 | 78 | 56 |
| 14 | 31 | 71 | 47 |
| 19 | 25 | 64 | 40 |
| 24 | 20 | 58 | 35 |
| 30 | 16 | 52 | 31 |

### EXAMPLE 3

Component A (OH component):

| Weight (g) | Product | Description |
|---|---|---|
| 104 | Desmophen 5034 BT | Trihunctional polypropylene |
| | (Bayer AG) | ether polyol |
| 181.2 | Baycoll VP KA 8576 | Polyester-polyol |
| | (Bayer AG) | |

A formulation having the following composition is prepared:

| Weight (g) | Product | Description |
|---|---|---|
| 0.3 | PLB-1 | Photolatent base |
| 2 | Butyl Acetate (Aldrich) | Solvent |
| 5 | Component A | OH component |
| 5 | Desmodur E 23 (Bayer AG) | Aromatic polyisocyanate pre- |
| | | polymer |

PLB-1 is dissolved in the component A and the isocyanate is added shortly before application.

A 100 μm thick film is applied onto a glass plate (plate A). The film is dried for 10 minutes at 40° C. A second glass plate (plate B), not coated with the adhesive, is pressed on plate A. After laminating plate A and plate B, the system is exposed to UV light (medium pressure mercury lamp from 1ST, one pass at a belt speed of 5 m/min with 2 lamps at 80 W/cm). 30 minutes after irradiation, it is no more possible to separate both glass plates. As a comparison, the same experiment is repeated with a system stored for 30 minutes in the dark instead of being irradiated. Both glass plates can be easily detached, the formulation being still liquid.

### EXAMPLE 4

A composition with the following ingredients is prepared:

| Weight (g) | Product | Description |
|---|---|---|
| 0.2 g | photoinitiator to be tested | Photolatent base |
| 13.3 g | Trimethylolpropane tris(3- | Thiol component |
| | mercaptopropionate) (Aldrich) | |
| 25 g | Tolonate HDT LV (Rhodia) | Isocyanate component |

The photolatent base is dissolved in the thiol-component and the isocyanate is added shortly before application.

Samples with PLB-2, PLB-3, PLB-4 and PLB-5 are prepared.

A 100 μm thick film is applied onto a glass plate (plate A). The film is dried for 10 minutes at 40° C. A second glass plate (plate B), not coated with the adhesive, is pressed on plate A. After laminating plate A and plate B, the samples are exposed for 5 minutes to a fluorescent lamp (Philips TL40W/05).

After irradiation, with all samples it is no more possible to separate both glass plates. As a comparison, the same experiment is repeated with a system stored for 5 minutes in the dark instead of being irradiated. Both glass plates can be easily detached, the formulation being still liquid.

### EXAMPLE 5

A composition with the following ingredients is prepared:

| Weight (g) | Product | Description |
|---|---|---|
| 0.2 g | PLB-1 | Photolatent base |
| 13.3 g | Trimethylolpropane tris(3- | Thiol component |
| | mercaptopropionate) (Aldrich) | |
| 25 g | Tolonate HDT LV (Rhodia) | Isocyanate component |

PLB-1 is dissolved in the thiol component and the isocyanate is added shortly before application.

A 100 μm thick film is applied onto an opaque substrate (plate A). The film is dried for 10 minutes at 40° C. The system is exposed for 1 minute to a fluorescent lamp (Philips TL40W/05). Immediately after exposure, a second opaque substrate (plate B), not coated with the adhesive, is pressed on plate A. After 10 seconds, it is no more possible to separate both plates.

### EXAMPLE 6

Component A (OH component):

| Weight (g) | Product | Description |
|---|---|---|
| 104 | Desmophen 5034 BT | Trifunctional polypropylene |
| | (Bayer AG) | ether polyol |
| 181.2 | Baycoll VP KA 8576 | Polyester-polyol |
| | (Bayer AG) | |

A formulation having the following composition is prepared:

| Weight (g) | Product | Description |
|---|---|---|
| 0.3 | PLB-6 | Photolatent base |
| 0.3 | DAROCUR ® ITX | Sensitizer |
| | (Ciba specialty Chemicals) | (isopropylthioxanthone) |
| 2 | Butyl Acetate (Aldrich) | Solvent |
| 5 | Component A | OH component |
| 5 | Desmodur E 23 (Bayer AG) | Aromatic polyisocyanate pre- |
| | | polymer |

PLB-6 and the sensitizer are dissolved in the component A and the isocyanate is added shortly before application.

A 100 μm thick film is applied onto a glass plate (plate A). The film is dried for 10 minutes at 40° C. A second glass plate (plate B), not coated with the adhesive, is pressed on plate A. After laminating plate A and plate B, the system is exposed to UV light (medium pressure mercury lamp from 1ST, one pass at a belt speed of 5 m/min with 2 lamps at 80 W/cm). 100 minutes after irradiation, it is no more possible to separate both glass plates.

1. A method of bonding a first substrate to a second substrate, comprising the steps of
a) applying a UV-curable adhesive resin composition comprising a photolatent base to at least one transparent surface of at least one of said first and second substrates,
b) bringing said first and second substrates together with said adhesive composition there between,
c) exposing said adhesive composition to actinic radiation to effect curing.

2. A method of bonding a first substrate to a second substrate, comprising the steps of
a) applying a UV-curable adhesive resin composition comprising a photolatent base to one surface,
b) exposing said adhesive composition to actinic radiation to effect curing,
c) bringing said first and second substrates together with said adhesive composition there between.

3. A method according to claim 1, wherein the photolatent base is a compound of the formula (I) 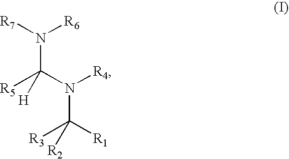
in which
R₁ is phenyl, biphenylyl, naphthyl, anthryl or anthraquinonyl all of which are unsubstituted or substituted by one or more of the substituents C₁-C₄-alkyl, C₂-C₄-alkenyl, CN, OR₁₀, SR₁₀, COOR₁₂, halogen or a substituent of structure (II) 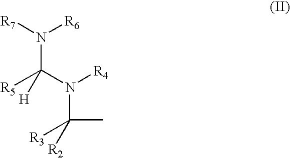
or
R₁ is a substituent of formula (IIIa) or (IIIb) 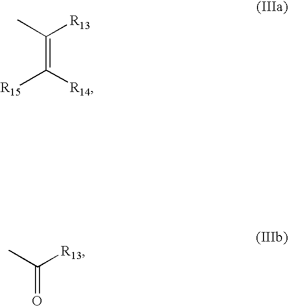
in which
R₁₃ is phenyl, biphenylyl, naphthyl, anthryl or anthraquinonyl all of which are unsubstituted or substituted by one or more of the substituents C₂-C₄-alkenyl, CN, OR₁₀, SR₁₀, COR₁₁, COOR₁₂, or halogen;
R₁₄ is hydrogen
R₁₅ is hydrogen or C₁-C₄-alkyl;
R₂ and R₃ independently of each other are hydrogen or C₁-C₆-alkyl;
R₄ and R₆ together form a C₂-C₆-alkylene bridge that is unsubstituted or substituted by one or more C₁-C₄-alkyl; or
R₅ and R₇, together form a C₂-C₆-alkylene bridge that is unsubstituted or substituted by one or more C₁-C₄-alkyl;
R₁₀, R₁₁ and R₁₂ independently of each other are hydrogen or C₁-C₆-alkyl.

4. A method according to claim 3 wherein the photolatent base is a compound 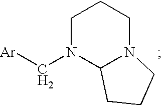
wherein
Ar is phenyl, biphenylyl or naphthyl all of which are unsubstituted or substituted by one or more of the substituents C₁-C₄-alkyl, CN, OH, O—C₁-C₆alkyl, SH, S—C₁-C₆alkyl or COOH, COO—C₁-C₆alkyl.

5. A method according to claim 4, wherein the photolatent base is

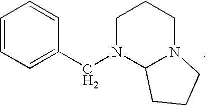

6. A method according to claim 3, wherein the photolatent base is a compound of formula I wherein R₁ is a substituent of formula III.

7. A method according to claim 6 wherein the photolatent base is

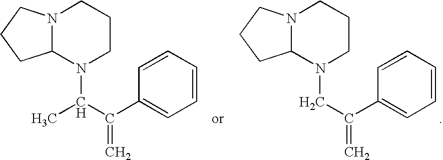

8. A method according to claim 1, wherein the photolatent base is a compound of the formula IV 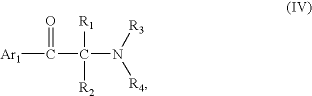
in which
Arₜ is an aromatic radical of formula V or VIII 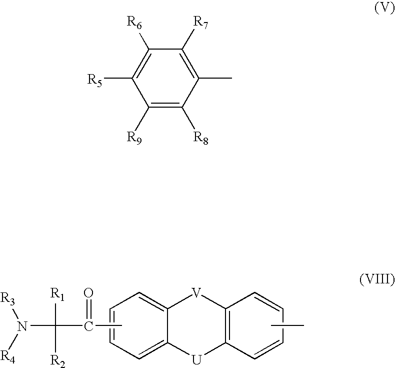
U is N(R₁₇)—;
V has the meaning of U or is a direct bond;
R₁ and R₂ are each independently of each other
a) C₁-C₁₂-alkyl, which is unsubstituted or substituted by OH, C₁-C₄-alkoxy, or SH,
b) a radical of formula 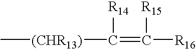
or
c) a radical of formula in which q is 0, or 1, 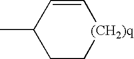
or
d) a radical of formula 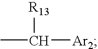
e) phenyl which is unsubstituted or substituted C₁-C₄-alkyl;
or R₁ and R₂ together are unbranched or branched C₄-C₆-alkylene or C₃-C₅-oxaalkylene,
Ar₂ is phenyl which is unsubstituted or substituted by halogen, OH, C₁-C₁₂-alkyl, or is substituted by C₁-C₄-alkyl, which is substituted by OH, halogen, C₁-C₁₂-alkoxy, —COO(C₁-C₄-alkyl), —CO(OCH₂CH₂)ₙOCH₃ or —COO(C₁-C₄-alkyl), or the radical phenyl, is substituted by C₁-C₄-alkoxy, —(OCH₂CH₂)ₙOH, or —(OCH₂CH₂)ₙOCH₃;
n is 1-5
R₃ is C₁-C₄-alkyl, C₂-C₄-alkyl which is substituted by —OH, —C₁-C₄-alkoxy, —CN, or —COO(C₁-C₄-alkyl), or R₃ is C₃-C₅-alkenyl, or phenyl-C₁-C₃-alkyl-;
R₄ is C₁-C₄-alkyl, C₂-C₄-alkyl which is substituted by —OH, —C₁-C₄-alkoxy, —CN, or —COO(C₁-C₄-alkyl), or R₃ is C₃-C₅-alkenyl, or phenyl-C₁-C₃-alkyl, or R₃ and R₄ together are C₃-C₇-alkylene which optionally is interrupted by —O—, or —S—;
R₅, R₆, R₇, R₈ and R₉ are each independently of one another hydrogen, halogen, C₁-C₁₂-alkyl, phenyl, benzyl, benzoyl, or a group —OR₁₇, —SR₁₅, —N(R₁₉)(R₂₀), or are 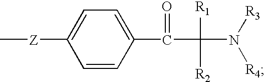
Z is —O—, —S—, —N(R₁₁)—, —N(R₁₁)—R₁₂—N(R₁₁)— or 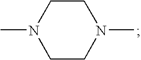
R₁₁ is C₁-C₄-alkyl;
R₁₂ is unbranched or branched C₂-C₁₆-alkylene which can be interrupted by one or more —O— or —S—;
R₁₃ is hydrogen or C₁-C₄-alkyl;
R₁₄, R₁₅ and R₁₆ are each independently of one another hydrogen or C₁-C₄-alkyl, or R₁₄ and R₁₅ together are C₃-C₄-alkylene;
R₁₇ is hydrogen, C₁-C₁₂-alkyl, C₃-C₆-alkenyl, C₂-C₆-alkyl which is substituted by —CN, —OH or —COO(C₁-C₄-alkyl);
R₁₈ is hydrogen, C₁-C₁₂-alkyl, C₃-C₆-alkenyl, C₂-C₁₂-alkyl which is substituted by —OH, CN, —COO(C₁-C₄-alkyl);
R₁₉ and R₂₀ are each independently of the other C₁-C₆-alkyl, C₂-C₄-hydroxyalkyl, C₂-C₁₀-alkoxyalkyl, C₃-C₅-alkenyl, phenyl-C₁-C₃-alkyl, phenyl which is unsubstituted or substituted by C₁-C₄-alkyl or C₁-C₄-alkoxy, or R₁₉ and R₂₀ are C₂-C₃-alkanoyl or benzoyl, or R₁₉ and R₂₀ are —O(CO—C₁-C₈)ₒ—OH;
o is 1-15;
or R₁₉ and R₂₀ together are C₄-C₆-alkylene which can be interrupted by —O—, —N(R₂₂)— or —S—, or R₁₉ and R₂₀ together are C₄-C₆-alkylene which can be substituted by hydroxyl, C₁-C₄-alkoxy or —COO(C₁-C₄-alkyl);
R₂₂ is C₁-C₄-alkyl, phenyl-C₁-C₃-alkyl, —CH₂CH₂—COO(C₁-C₄-alkyl), —CH₂CH₂CN, —CH₂CH₂— 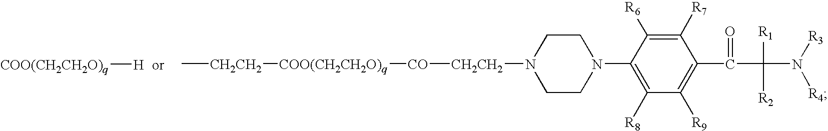
and q is 1-8.

9. A method according to claim 8, wherein the photolatent base is 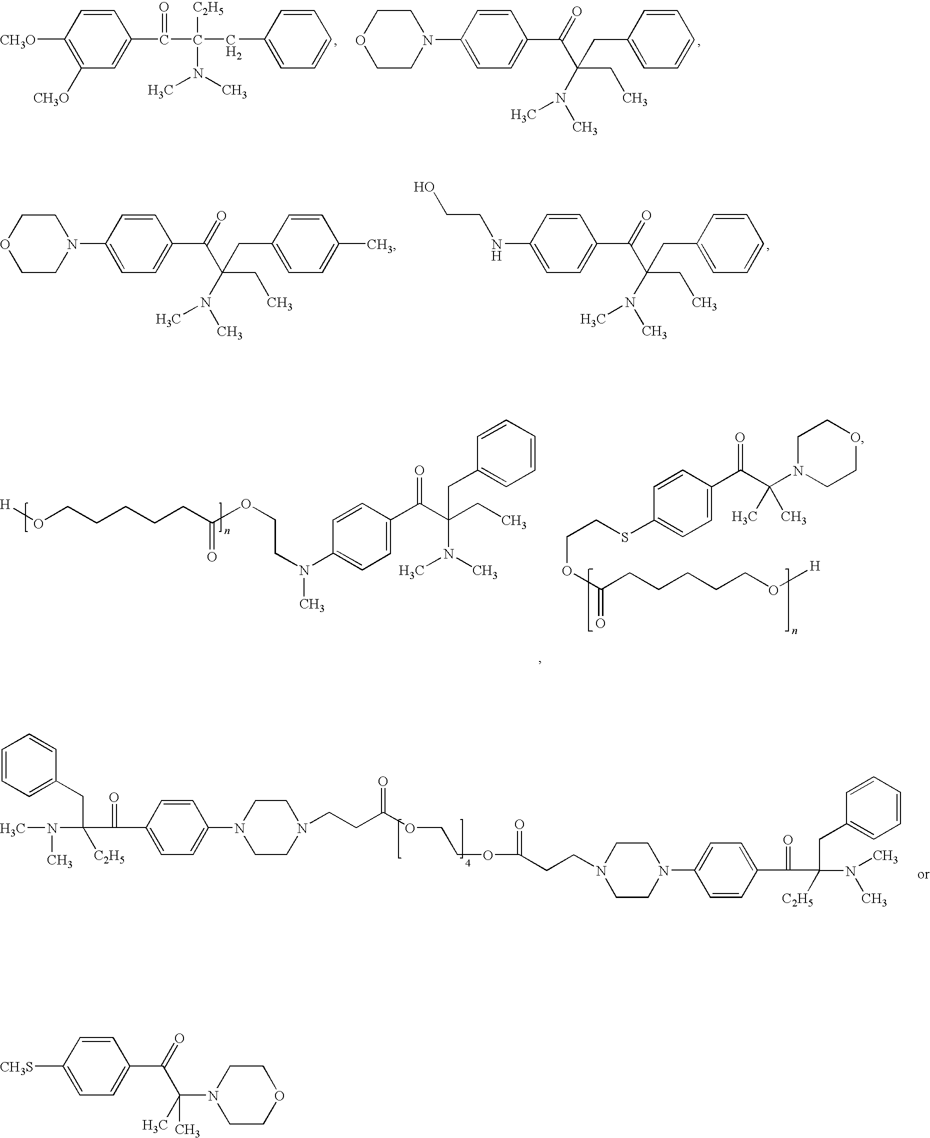
with n=0-10.

10. A method according to claim 1 wherein the UV-curable adhesive resin composition comprises a polyurethane adhesive SH/NCO resin and the photolatent base is

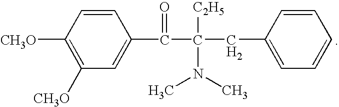

11. A UV-curable adhesive resin composition comprising 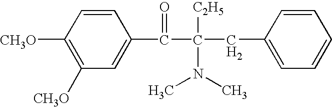
as photolatent base in an adhesive.

12. A method according to claim 2, wherein the photolatent base is a compound of the formula (I) 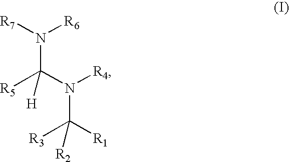
in which
R₁ is phenyl, biphenylyl, naphthyl, anthryl or anthraquinonyl all of which are unsubstituted or substituted by one or more of the substituents C₁-C₄-alkyl, C₂-C₄-alkenyl, CN, OR₁₀, SR₁₀, COOR₁₂, halogen or a substituent of structure (II) 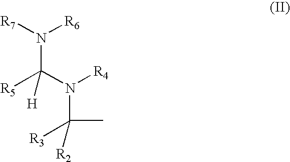
or
R₁ is a substituent of formula (IIIa) or (IIIb) 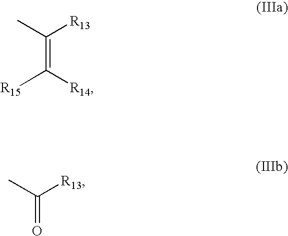
in which
R₁₃ is phenyl, biphenylyl, naphthyl, anthryl or anthraquinonyl all of which are unsubstituted or substituted by one or more of the substituents C₁-C₄-alkyl, C₂-C₄-alkenyl, CN, OR₁₀, SR₁₀, COR₁₁, COOR₁₂, or halogen;
R₁₄ is hydrogen
R₁₅ is hydrogen or C₁-C₄-alkyl;
R₂ and R₃ independently of each other are hydrogen or C₁-C₆-alkyl;
R₄ and R₆ together form a C₂-C₆-alkylene bridge that is unsubstituted or substituted by one or more C₁-C₄-alkyl; or
R₅ and R₇, together form a C₂-C₆-alkylene bridge that is unsubstituted or substituted by one or more C₁-C₄-alkyl;
R₁₀, R₁₁ and R₁₂ independently of each other are hydrogen or C₁-C₆-alkyl.

13. A method according to claim 12 wherein the photolatent base is a compound 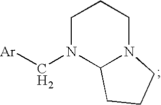
wherein
Ar is phenyl, biphenylyl or naphthyl all of which are unsubstituted or substituted by one or more of the substituents CN, OH, O—C₁-C₆alkyl, SH, S—C₁-C₆alkyl or COOH, COO—C₁-C₆alkyl.

14. A method according to claim 13, wherein the photolatent base is

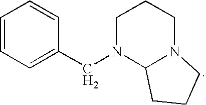

15. A method according to claim 12, wherein the photolatent base is a compound of formula I wherein R₁ is a substituent of formula III.

16. A method according to claim 15 wherein the photolatent base is

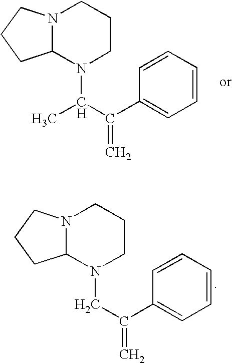

17. A method according to claim 2, wherein the photolatent base is a compound of the formula IV 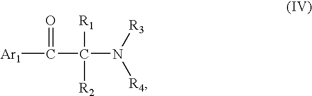
in which
Ar₁ is an aromatic radical of formula V or VIII 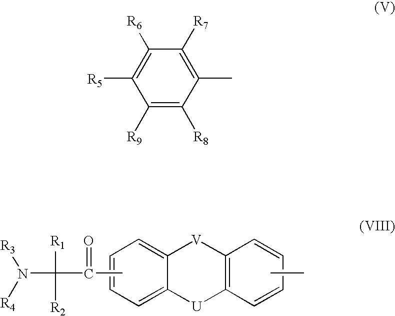
U is N(R₁₇)—;
V has the meaning of U or is a direct bond;
R₁ and R₂ are each independently of each other
f) C₁-C₁₂-alkyl, which is unsubstituted or substituted by OH, C₁-C₄-alkoxy, or SH,
g) a radical of formula 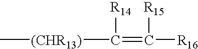
or
h) a radical of formula 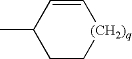
in which q is 0, or 1, or
i) a radical of formula 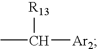
j) phenyl which is unsubstituted or substituted C₁-C₄-alkyl;
or R₁ and R₂ together are unbranched or branched C₄-C₆-alkylene or C₃-C₅-oxaalkylene,
Ar₂ is phenyl which is unsubstituted or substituted by halogen, OH, C₁-C₁₂-alkyl, or is substituted by C₁-C₄-alkyl, which is substituted by OH, halogen, C₁-C₁₂-alkoxy, —COO(C₁-C₄-alkyl), —CO(OCH₂CH₂)ₙOCH₃ or —COO(C₁-C₄-alkyl), or the radical phenyl, is substituted by C₁-C₄-alkoxy, —(OCH₂CH₂)ₙOH, or —(OCH₂CH₂)ₙOCH₃;
n is 1-5
R₃ is C₁-C₄-alkyl, C₂-C₄-alkyl which is substituted by —OH, —C₁-C₄-alkoxy, —CN, or —COO(C₁-C₄-alkyl), or R₃ is C₃-C₅-alkenyl, or phenyl-C₁-C₃-alkyl-;
R₄ is C₁-C₄-alkyl, C₂-C₄-alkyl which is substituted by —OH, —C₁-C₄-alkoxy, —CN, or —COO(C₁-C₄-alkyl), or R₃ is C₃-C₅-alkenyl, or phenyl-C₁-C₃-alkyl, or R₃ and R₄ together are C₃-C₇alkylene which optionally is interrupted by —O—, or —S—;
R₅, R₆, R₇, R₈ and R₉ are each independently of one another hydrogen, halogen, C₁-C₁₂-alkyl, phenyl, benzyl, benzoyl, or a group —OR₁₇, —SR₁₈, —N(R₁₉)(R₂₀), or are 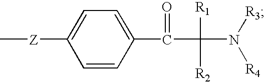
Z is —O—, —S—, —N(R₁₁)—, —N(R₁₁)—R₁₂—N(R₁₁)— or 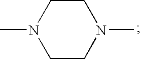
R₁₁ is C₁-C₄-alkyl;
R₁₂ is unbranched or branched C₂-C₁₆-alkylene which can be interrupted by one or more —O— or —S—;
R₁₃ is hydrogen or C₁-C₄-alkyl;
R₁₄, R₁₅ and R₁₆ are each independently of one another hydrogen or C₁-C₄-alkyl, or R₁₄ and R₁₅ together are C₃-C₄-alkylene;
R₁₇ is hydrogen, C₁-C₁₂-alkyl, C₃-C₆-alkenyl, C₂-C₆-alkyl which is substituted by —CN, —OH or —COO(C₁-C₄-alkyl);
R₁₈ is hydrogen, C₁-C₁₂-alkyl, C₃-C₆-alkenyl, C₂-C₁₂-alkyl which is substituted by —OH, CN, —COO(C₁-C₄-alkyl);
R₁₉ and R₂₀ are each independently of the other C₁-C₆-alkyl, C₂-C₄-hydroxyalkyl, C₂-C₁₀-alkoxyalkyl, C₃-C₅-alkenyl, phenyl-C₁-C₃-alkyl, phenyl which is unsubstituted or substituted by C₁-C₄-alkyl or C₁-C₄-alkoxy, or R₁₉ and R₂₀ are C₂-C₃-alkanoyl or benzoyl, or R₁₉ and R₂₀ are —O(CO—C₁-C₈)ₒ—OH;
o is 1-15;
or R₁₉ and R₂₀ together are C₄-C₆-alkylene which can be interrupted by —O—, —N(R₂₂)— or —S—, or R₁₉ and R₂₀ together are C₄-C₆-alkylene which can be substituted by hydroxyl, C₁-C₄-alkoxy or —COO(C₁-C₄-alkyl);
R₂₂ is C₁-C₄-alkyl, phenyl-C₁-C₃-alkyl, —CH₂CH₂—COO(C₁-C₄-alkyl), —CH₂CH₂CN, —CH₂CH₂—COO(CH₂CH₂O)_{q}—H or 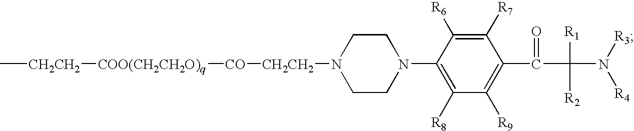
and
q is 1-8.

18. A method according to claim 17, wherein the photolatent base is 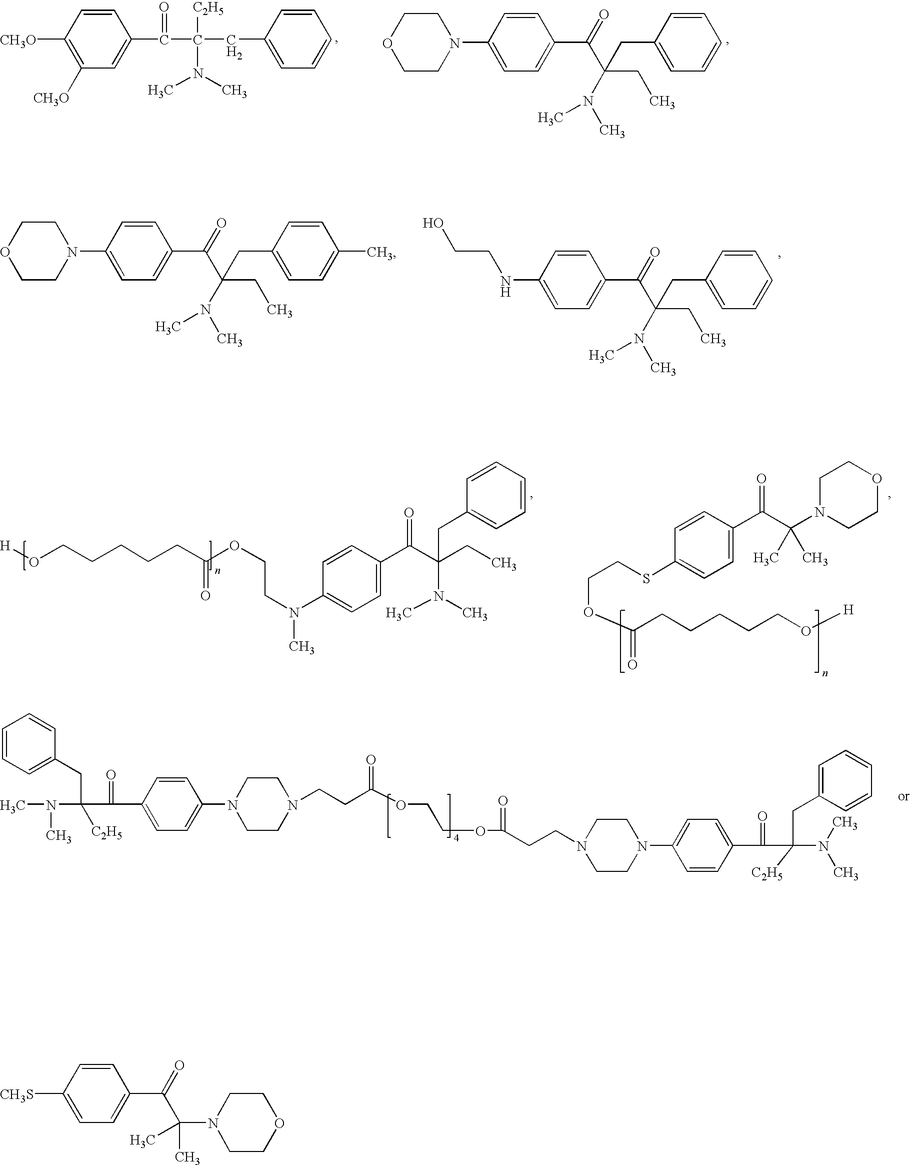
with n=0-10.

19. A method according to claim 2 wherein the UV-curable adhesive resin composition comprises a polyurethane adhesive SH/NCO resin and the photolatent base is [chemical structure]
